(19) United States
(12) Patent Application Publication
(10) Pub. No.: US 2026/0325103 A1
(43) Pub. Date: Oct. 22, 2026

(54) PROBE-HUMAN INTERFACE PROVIDING A PLACEHOLDER FOR AN ULTRASOUND PROBE ON A PATIENT

(71) Applicant: EchoNous, Inc., Redmond, WA (US)

(72) Inventors: Lazaro E. Hernandez, Cooper City, FL (US); Dallas Erdahl, Maple Grove, MN (US)

(21) Appl. No.: 19/649,952

(22) Filed: Apr. 16, 2026

Related U.S. Application Data
(60) Provisional application No. 63/790,530, filed on Apr. 17, 2025.

(51) Int. Cl. A61B 8/00

(52) U.S. Cl. CPC A61B 8/4444; A61B 8/4236

(57) ABSTRACT

A probe-human interface includes a base with an attachment mechanism that secures the base to the body of a patient. A frame connected to the base has a center opening that receives an ultrasound probe and provides access to the patient for communicating ultrasound signals. When the ultrasound probe is placed into the frame, a clip receives a portion of the ultrasound probe and secures the ultrasound probe to the frame. The clip may be a flexible ratchet clip coupled to the base at an angle that facilitates an engagement between the clip and the ultrasound probe at a desired position. Also described is an ultrasound system including an ultrasound probe and a probe-human interface, and an ultrasound method. The probe-human interface is affixed to the patient and receives the ultrasound probe in a releasable engagement whereby the ultrasound probe is secured in a consistent position relative to the patient.

### FIELD

This disclosure relates to wearable devices for inpatient and ambulatory care and telemedicine, for obtaining consistent ultrasound imaging and/or therapy of a patient.

### BACKGROUND

Ultrasound imaging is a valuable, non-invasive procedure for visualizing structures internal to a body. In particular, echocardiograms, also known as cardiac ultrasounds, involve an ultrasound probe configured for non-invasive medical imaging that allows the heart and nearby structures of a patient to be visualized, e.g., on a monitor or display screen. Cardiac ultrasounds are typically performed by trained cardiac sonographers or specialized cardiovascular medical providers.

It is challenging to properly position an ultrasound probe on a patient to obtain specific diagnostic ultrasound views. The probe is placed in contact with the patient’s skin and adjacent to a structure or region of interest to be visualized, such as the heart. Proper probe placement generally requires extensive training and experience.

### BRIEF SUMMARY

Systems and methods described herein enable ultrasound images to be obtained without requiring trained sonographers or specialized personnel to place the ultrasound probe each time that imaging is desired. A system described herein may be configured for any healthcare provider, patient, or caregiver to place an ultrasound imaging probe on the patient and consistently obtain desired ultrasound images. Systems described herein may also facilitate ultrasound imaging during a medical procedure.

Various embodiments of the disclosure provide a probe-human interface configured to receive and hold an ultrasound probe in relation to a patient. The probe has an ultrasound transducer for imaging the patient. The probe-human interface is positioned on the patient such that, when an ultrasound probe is coupled to the probe-human interface, the ultrasound transducer holds the probe in the proper position for consistent ultrasound imaging of the patient. Interaction between the probe-human interface and the ultrasound probe facilitates the fastening of the probe to a patient's body in a consistent manner without specialized tools or assistance from trained personnel. The probe-human interface allows a non-trained healthcare provider, caregiver, or patient to position and secure the ultrasound probe to the patient's body to obtain the desired imaging.

A trained healthcare provider generally first places the probe-human interface with an ultrasound probe on the patient's skin. After positioning the probe-human interface and obtaining a desired ultrasound (e.g., echocardiographic) view using the ultrasound probe, the healthcare provider secures the probe-human interface to the patient using an attachment mechanism, such as an adhesive. The ultrasound probe may be removed while the probe-human interface remains affixed to the patient. At a subsequent time, the ultrasound probe may be reattached to the probe-human interface and be properly positioned to again obtain the desired ultrasound view of the patient without requiring medical expertise to position the probe.

The probe-human interface is thus a mechanical device attached to the patient's body adjacent to the target examination area and configured to receive and hold the ultrasound probe in a consistent position. An ultrasound probe is designed not only to send and receive ultrasound signals to and from the patient's body, but also to mechanically interact with the probe-human interface so that the probe may be secured properly with respect to the patient’s body. Consequently, when the probe-human interface is attached to the patient adjacent to the target examination area, the probe-human interface functions as a placeholder for the ultrasound probe, allowing subsequent consistent acquisition of an ultrasound image as many times as the ultrasound probe is engaged with the probe-human interface on the patient’s body. Advantageously, the acquired image view and quality is improved while facilitating easier attachment and removal of the ultrasound probe from the patient.

### BRIEF DESCRIPTION OF THE SEVERAL VIEWS OF THE DRAWINGS

FIG. 1 is a top, front, right perspective view of one example of a probe-human interface of the present disclosure.

FIG. 2 is a top, front, left perspective view of one example of an ultrasound probe usable with the probe-human interface of FIG. 1.

FIG. 3 is a top, front, right perspective view of the probe-human interface engaged with the ultrasound probe in a first position.

FIG. 4 is a top, front, right perspective view of the probe-human interface engaged with the ultrasound probe in a second position.

FIG. 5 is a graphical depiction of an environment in which the probe-human interface is attached to a patient's body adjacent to a target examination area and acting as a placeholder for an ultrasound probe.

FIG. 6 is a graphical depiction of the environment shown in FIG. 5 in which an ultrasound probe is engaged with the probe-human interface.

FIG. 7 is a top, front, right perspective view of another example of a probe-human interface of the present disclosure.

FIG. 8 is a top, rear, right perspective view of the probe-human interface shown in FIG. 7.

FIG. 9 is a right side elevation view of the probe-human interface shown in FIG. 7.

FIG. 10 is a bottom plan view of the probe-human interface shown in FIG. 7.

FIG. 11 is a rear elevation view of the probe-human interface shown in FIG. 7.

FIG. 12 is an exploded top, rear, right perspective view of the probe-human interface shown in FIG. 7 with materials forming the base.

### DETAILED DESCRIPTION

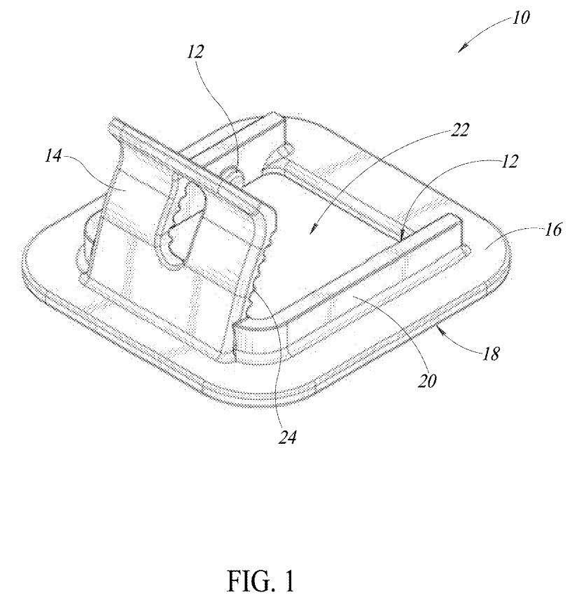

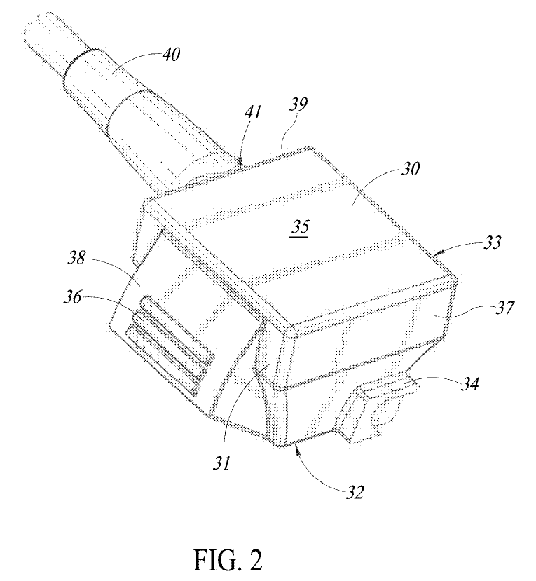

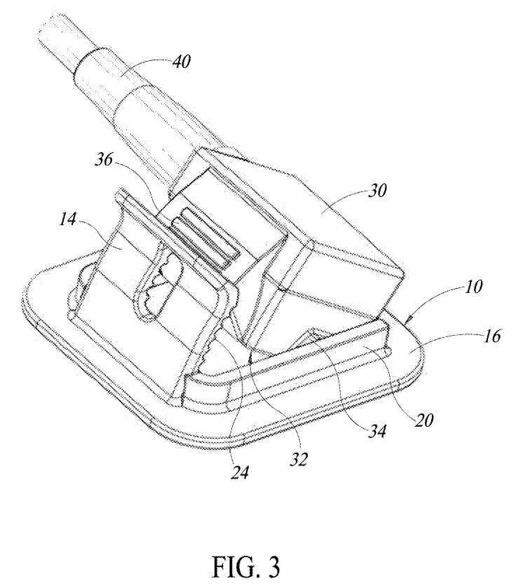

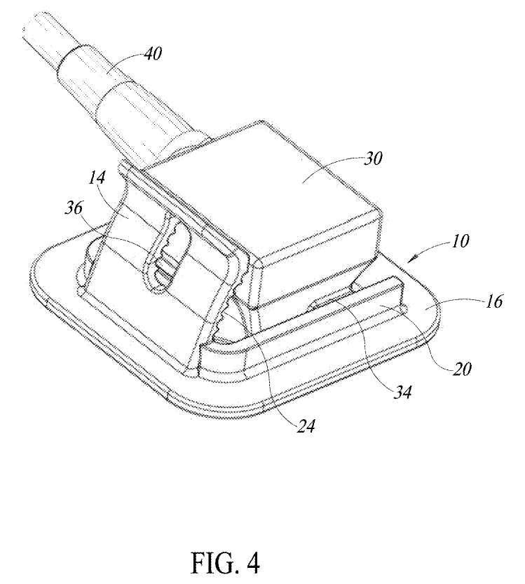

Systems and methods described herein facilitate consistent positioning of an ultrasound probe on a patient for ultrasound imaging, such as echocardiographic imaging of a patient. As can be seen in FIGS. 1-6, an exemplary system includes a probe-human interface 10 configured to receive and secure an ultrasound probe 30 to a patient’s body 50. An ultrasound probe 30, when attached to the probe-human interface 10, positions an ultrasound transducer 32 of the probe with respect to a region of interest in the patient 50. The ultrasound transducer 32 emits ultrasonic signals towards the patient 50 and receives echo signals in return. The echo signals are processed to produce an ultrasound image of structures within the patient’s body.

The probe-human interface 10 is a portable structure configured to act as a placeholder for the ultrasound probe 30 and, when secured to a patient 50, provides consistent positioning of the ultrasound probe 30 with respect to the patient 50. In the illustrated embodiment, the probe-human interface 10 includes two pins (hinge points) 12 and a flexible ratchet clip 14 that enables the ultrasound probe 30 to be attached to and removed from the probe-human interface 10. The probe-human interface 10 enables repeatable attachment and removal of the ultrasound probe 30 while the probe-human interface 10 remains affixed to the patient 50.

By acting as a placeholder, the probe-human interface 10 enables the ultrasound probe 30 to be securely attached to the patient's body adjacent to a target examination area. In the embodiment shown in FIGS. 1-6, the probe-human interface 10 includes a base 16 and an attachment mechanism 18 that attaches the probe-human interface 10 to the patient. The attachment mechanism 18 may be, for example, an adhesive arranged on or around the base 16 that, when exposed to the patient, affixes the base 16 to the patient. In some embodiments, the adhesive may be a flexible adhesive tape secured along or around one or more edges of the base 16. In other embodiments, the attachment mechanism may be, for example, a belt that is coupled to the base 16, wherein the belt is worn by the patient to affix the base 16 to the patient.

Connected to the base 16 is a frame 20 having a center opening 22. The frame 20 and the center opening 22 are sized to receive the ultrasound probe 30 and position the ultrasound probe 30 with respect to a target examination area of the patient. In the illustrated embodiment, the frame 20 and the center opening 22 are rectangular, though the frame 20 and the center opening 22 may each have any suitable shape or dimension. In various embodiments, the frame 20 may be integral with the base 16 or it may be coupled to the base 16 by affixing the frame 20 to the base 16.

In use, the base 16 is placed in contact with the patient, typically in direct contact with the patient’s skin, and is secured to the body of the patient by the attachment mechanism 18 (e.g., adhesive) to the patient. The center opening 22 provides an area or space within the frame 20 for an ultrasound probe 30 to access the patient when the ultrasound probe 30 is coupled to the probe-human interface 10. Through the center opening 22, the ultrasound transducer 32 of the probe 30 achieves an acoustic coupling with the patient to effectively transmit ultrasound signals into the patient and receive echo signals in return.

In the illustrated embodiment, the probe-human interface 10 has a clip 14 that releasably secures an ultrasound probe 30 to the base 16. The clip 14 may be a flexible ratchet clip as shown, though other mechanisms may be employed to releasably secure an ultrasound probe to the base 16. The flexible ratchet clip 14, working in cooperation with the hinge points 12, provides a safe, secure, and releasable attachment of the ultrasound probe 30 to the probe-human interface 10 and thus, in turn, a releasable attachment of the ultrasound probe 30 to the patient 50.

In various embodiments, the flexible ratchet clip 14 of the probe-human interface 10 is oriented cephalad or away from the base 16 at an angle that facilitates an engagement between the clip 14 and the ultrasound probe 30. The clip 14 includes one or more laterally-extending rachet elements or pawl teeth 24 that are configured to engage with corresponding ridges or gear teeth 36 on a gear protrusion 38 of the ultrasound probe 30.

When the probe-human interface 10 is used for remote ultrasound examinations, for example, the probe-human interface 10 can be affixed to the patient and comfortably worn under clothing when not in use.

The ultrasound probe includes a probe housing, internal components, and an electrical (e.g., coaxial) cable 40. The cable 40 delivers power to the ultrasound probe 30 and may support communication of signals to and from the ultrasound probe 30. The probe housing comprises a front section 31, a rear section 33, an upper section 35, a first lateral section 37, and a second lateral section 39 , as well as a wiring opening 41 and probe opening on a lower section where the ultrasound transducer 32 is located. The front section 31, rear section 33, upper section 35, first lateral section 37, and second lateral section 39 correspond to the sides of the probe housing. Internally, the probe 30 includes components such as a processor and acoustic stack that, in combination with the ultrasound transducer 32, are configured to transmit and receive ultrasound signals to and from the patient. The wiring as shown corresponds to a coaxial cable 40 that connects the internal components with an external transducer controller. In some embodiments, the ultrasound probe 30 may be entirely self contained with a power source, controller, and wireless communication circuitry for receiving instructions and providing ultrasound imaging data as acquired by the ultrasound probe 30. While the figures depict the ultrasound transducer cable 40 and the probe housing (with the acoustic stack) at a 90-degree angle to each other, such orientation and connection may be adjusted according to specific implementations.

The probe-human interface 10 and the ultrasound probe 30 have a corresponding a hinge mechanism that in combination with the clip 14 provides a releasable attachment of the probe 30 to the base 16. In general, for the probe 30 as illustrated, the hinge mechanism includes a first slot 34 on the first lateral section 37 of the probe housing and a second slot similar to slot 34 on the second lateral section 39 opposite of the first slot 34. Correspondingly, the probe-human interface 10 includes a first pin 12 on one side of the frame 20 and a second pin similar to the first pin 12 on an opposite side of the frame 20. The first and second pins 12 may be configured as short cylindrical protrusions that are sized to slide into and engage the first and second slots 34, respectively. The first and second slots 34 are arranged such that each pin 12 can be inserted into the corresponding slot 34 so that the housing of the probe 30 can pivot on the base 16 and, in combination with the clip 14, secure the probe 30 to the base 16. In this manner, the first pin 12 is externally integrated into a first lateral side of the frame 20 while the second pin 12 is externally integrated into an opposite second lateral side of the frame 20. The first and second pins 12 are thus positioned opposite to each other across the frame 20. The first and the second slots 34 are positioned on the probe 30 adjacent to the lower section 32 and the rear section 33 of the probe housing. Whereas the first and second pins 12 are integrated into the base 16 within the center opening 22, the first and second slots 34 are positioned opposite to each other across the probe housing to match the arrangement of the first and second pins 12.

Further, the first and second pins 12 are positioned offset to the flexible ratchet clip 14. Thus, when a user (e.g., healthcare provider, patient, or caregiver) inserts the first and second pins 12 into the first and second slots 24, respectively, the housing of the probe 30 can be pivoted on the base 16 of probe-human interface 10, near the rear side.

Alternatively, the pins and slots described above may be switched between the probe-human interface 10 and the ultrasound probe 30. In at least one such alternative arrangement, the probe-human interface 10 includes a first slot on one side of the frame 20 and a second slot similar to the first slot on an opposite side of the frame 20. The first and second slots are sized to receive and engage first and second pins, respectively, defined on the ultrasound probe 30. The first and second slots are arranged such that each pin can be inserted into the corresponding slot and enable the housing of the probe 30 to pivot on the base 16 and, in combination with the clip 14, secure the probe 30 to the base 16.

It should further be understood that in other suitable embodiments, the hinge mechanism may be implemented differently yet provide the releasable attachment of the ultrasound probe 30 to the base 16 of the probe-human interface 16 as contemplated herein. Such different mechanism or mechanisms may be a different arrangement of pins and slots or other mechanisms that mate and allow the releasable attachment of the ultrasound probe 30 to the base 16.

As can be seen in FIGS. 1-6, the ratchet mechanism of the present disclosure may include a gear protrusion 38 and a corresponding flexible ratchet clip 14. In the illustrated embodiment, the gear protrusion 38 is arranged on the ultrasound probe 30 and the flexible ratchet clip 14 is arranged on the probe-human interface 10, though such an arrangement may be modified as desired to allow a selective positional engagement and securing of the ultrasound probe 30 with the frame 20 of the probe-human interface 10.

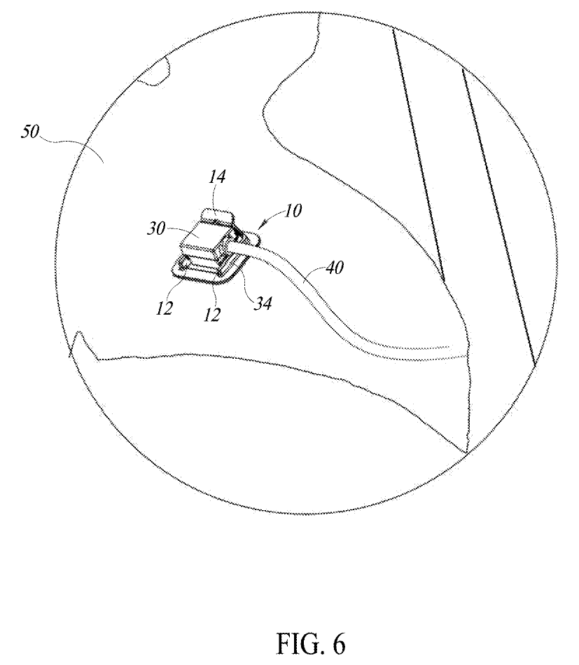

In the embodiment shown, the gear protrusion 38 is integrated into the front section 31 of the probe 30 and shaped to correspond with the shape of the clip 14 of the probe-human interface 10. The gear protrusion 38 includes a plurality of gear teeth 36 that are externally integrated into the gear protrusion 38. The plurality of gear teeth 36 are distributed along the gear protrusion 38. In a corresponding manner, the clip 14 of probe-human interface 10 includes a plurality of pawl teeth 24 that are distributed along the clip 14 and oriented towards the center opening 22 so that the plurality of pawl teeth 24 can engage the plurality of gear teeth 36 when the gear protrusion 38 of the probe 30 is positioned against the clip 14. The distribution of the plurality of pawl teeth 24 and the plurality of gear teeth 36 is arranged such that the orientation of the ultrasound probe 30 is adjustable when mounted onto the base 16. For example in use, once the ultrasound probe 30 is connected to the base 16 using the hinge mechanism described herein, the user (e.g., healthcare provider, patient, or caregiver) can press the housing of the probe 30 toward the base 16, with the ultrasound transducer 32 oriented towards the center opening 22. As the ultrasound transducer 32 gets closer to the patient's body, the user can continue pressing the probe housing until the desired orientation of the probe 30 is achieved. Once the desired orientation is achieved, the ratchet mechanism (i.e., the plurality of pawl teeth 24 and corresponding plurality of gear teeth 36) locks the probe 30 in place within the frame 20 and thus positioned with respect to the patient, e.g., patient 50 as shown in FIG. 6. In other suitable embodiments, different mechanisms can be utilized in the probe-human interface 10 to removably attach and secure the ultrasound probe 30 in place on the patient's body.

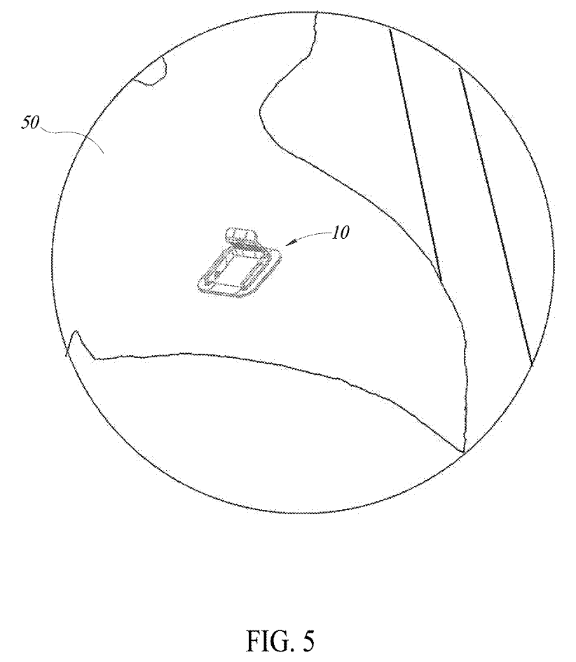

During an initial ultrasound procedure, an appropriate healthcare provider positions the probe-human interface 10 and probe 30 to obtain a desired ultrasound image of the patient 50. The probe-human interface 10 is affixed to the patient 50 adjacent to the target examination area, e.g., as shown in FIGS. 5 and 6. In this manner, the probe-human interface 10 is positioned and attached to the patient's body, allowing the probe-human interface 10 to serve as a placeholder for the ultrasound probe 30 when ultrasound imaging of the patient 50 is desired. The ultrasound probe 30 can be removed from and reattached to the probe-human interface 10 multiple times by non-trained individuals and the probe-human interface 10 is able to obtain consistent desired ultrasound images.

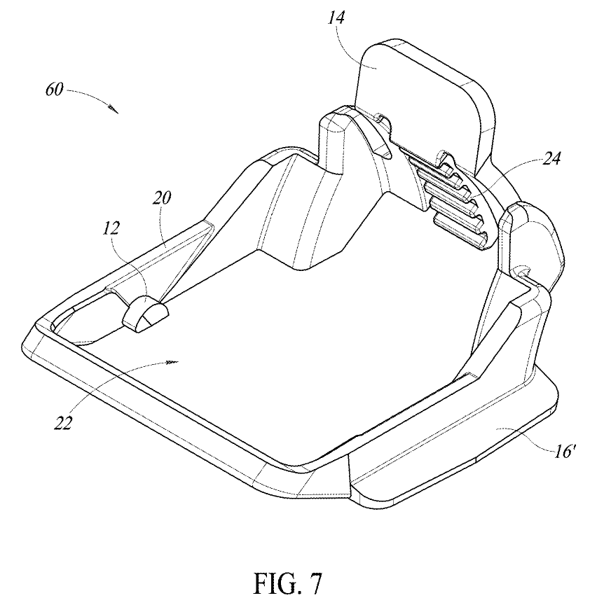

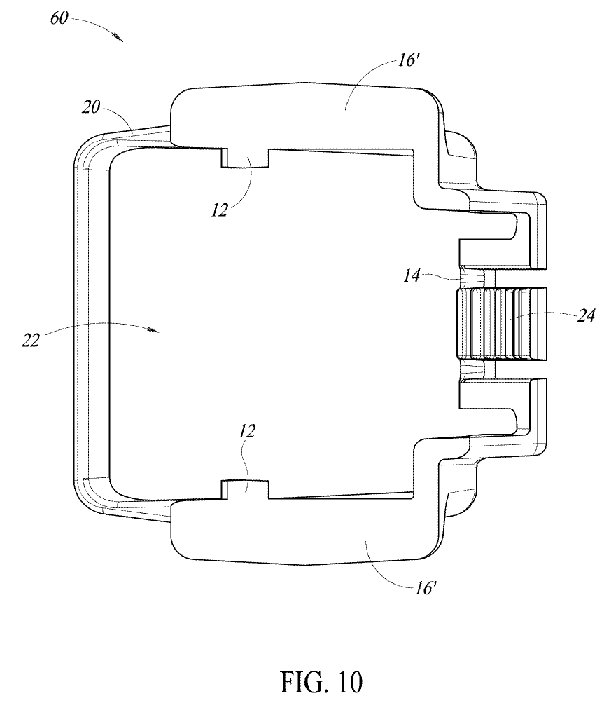

FIGS. 7-12 illustrate another example of a probe-human interface 60 according to the present disclosure. FIG. 7 is a top, front, right perspective view of the probe-human interface 60. Aspects of the probe-human interface 60 that are similar to or the same as aspects of the probe-human interface 10 shown in FIGS. 1-6 are indicated using like reference numerals. Thus, in FIG. 7 the probe-human interface 60 is shown including pins 12, a clip 14, a frame 20, and a center opening 22. FIG. 10, described below, further depicts the pins 12.

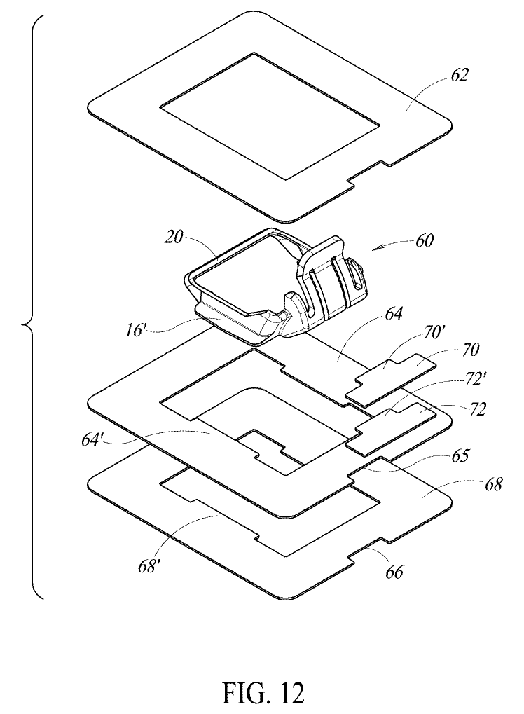

The probe-human interface 60 includes wings 16’ that laterally extend from a bottom edge of the frame 20. The wings 16’ may be integrally formed with the frame 20 as illustrated, or they may be separate elements that are connected to the frame 20. As will be described in greater detail below in regard to FIG. 12, the wings 16’ form part of a base 16 that is usable to affix the probe-human interface 60 to a patient, e.g., as shown in FIGS. 5 and 6.

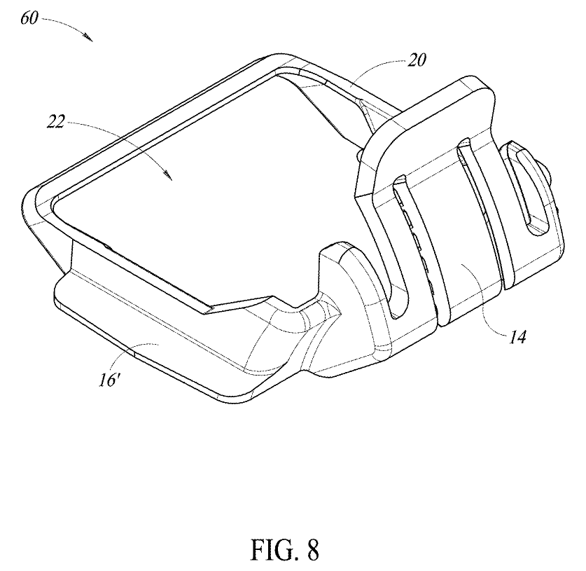

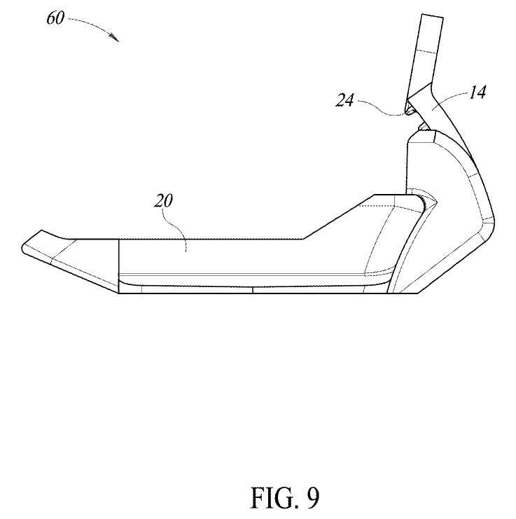

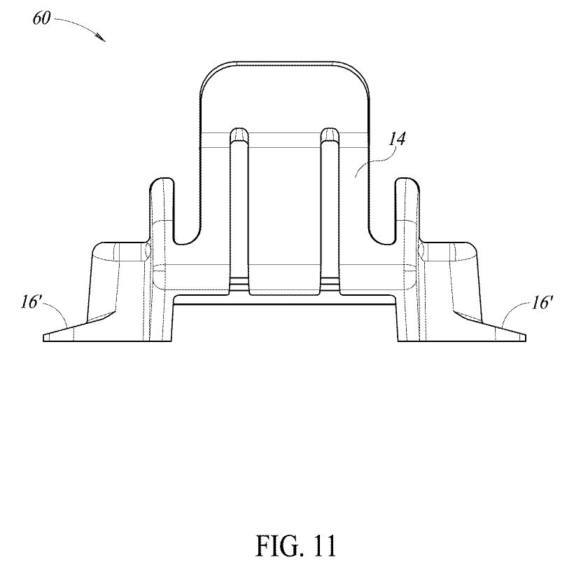

FIG. 8 is a top, rear, right perspective view of the probe-human interface 60 shown in FIG. 7, while FIG. 9 is a right side elevation view thereof, FIG. 10 is a bottom plan view thereof, and FIG. 11 is a rear elevation view thereof. FIGS. 9 and 10 also illustrate pawl teeth 24 on the clip 14 that are similarly depicted and described in regard to FIGS. 1, 3 and 4.

FIG. 12 is an exploded top, rear, right perspective view of the probe-human interface 60 shown in FIG. 7. Further depicted in FIG. 12 are materials 62, 64, and 68 that, when assembled, form the base 16 of the probe-human interface 60.

More specifically, the materials in FIG. 12 include a top surface 62, a bottom surface 64, and a removable liner 68. The top surface 62 is formed of suitable material such as a medical foam tape that is cut, e.g., by a die, and in assembly, fitted over or around the frame 20 of the probe-human interface 60. When assembled, the top surface 62 lies over the wings 16’ of the frame 20. Preferably the top surface 62 is attached, e.g., via an adhesive, to the wings 16’.

The bottom surface 64 is formed of a suitable material such as a medical tape having a double sided adhesive. Using such adhesive, when the base 16 is assembled, the top side of the bottom surface 64 adheres to the bottom side of the top surface 62 and underlies the wings 16’. The bottom surface 64 preferably has a periphery that matches the periphery of the top surface 62.

The bottom surface 64 extends under the wings 16’ of the frame 20 and preferably adheres to the wings 16’. In some cases, the bottom surface 64 includes inwardly extending tabs 64’ that further support the frame 20, preferably by attachment to the underside of the wings 16’. The wings 16’ are thus sandwiched between the top surface 62 and the bottom surface 64.

In a non-deployed state, the removable liner 68 is attached to the underside of the bottom surface 64. The removable liner 68 covers adhesive material on the underside of the bottom surface 64. When the probe-human interface 60 is to be deployed, the removable liner 68 is removed, thus exposing the adhesive underside of the bottom surface 64. In this state, the probe-human interface 60 is affixable to a patient, e.g., as depicted in FIGS. 5 and 6, by placing the probe-human interface 60 with the adhesive bottom surface 64 in a desired position on the patient. In cases where the bottom surface 64 has inwardly extending tabs 64’ as discussed above, the removable liner 68 preferably includes matching inwardly extending tabs 68’ that match the periphery of the bottom surface 64 and cover adhesive material on the tabs 64’ of the bottom surface 64.

The removable liner 68 may be constructed of suitable materials, such as a bleached kraft paper having a silicone layer on the top side of the removable liner 68. In some cases, the silicone layer may be provided on both the top and bottom sides of the removable liner 68. The silicone layer facilitates removal of the removable liner 68 from the adhesive underside of the bottom surface 64.

The bottom surface 64 and removable liner 68 are further depicted in FIG. 12 including notches 65 and 66, respectively. When assembled, the probe-human interface 60 may include additional templates 70 and 72 having inwardly extending tabs 70’ and 72’, respectively, that fit the notches 65 and 66, respectively. The templates 70 and 72 are arranged in the notches 65 and 66 to facilitate handling and positioning of the probe-human interface 60 when the probe-human interface is deployed on a patient. The templates 70 and 72 may be constructed of the same or similar materials as the bottom surface 64 and the removable liner 68, respectively.

Thus, as described herein, at least one example of a probe-human interface includes a base and a frame connected to the base. The base has an attachment mechanism configured to secure the base to a body of a patient. The frame has a center opening that is sized and arranged to receive an ultrasound probe and provide access to the patient for the ultrasound probe to send and receive ultrasound signals to and from the patient. The probe human interface also includes a clip that is configured to receive a portion of the ultrasound probe when the ultrasound probe is placed into the frame. The clip is configured to secure the ultrasound probe to the frame.

In various implementations, the probe-human interface may include any one or any combination of the following features, wherein: the clip is a flexible ratchet clip that is coupled to the base at an angle relative to the base, and the angle of the clip facilitates an engagement between the clip and the ultrasound probe when the ultrasound probe is placed into the frame; the clip is biased toward the center opening such that when the ultrasound probe is placed into the frame, the clip bears against a housing of the ultrasound probe; the clip has one or more laterally-extending ratchet elements that are configured to engage with a protrusion on the ultrasound probe; the one or more laterally-extending ratchet elements include pawl teeth that are configured to engage with corresponding gear teeth on the protrusion of the ultrasound probe; and/or the clip is configured to secure the ultrasound probe within the frame at a plurality of positions with respect to the base.

In some cases, the probe-human interface further includes a hinge mechanism coupled to the frame. The hinge mechanism in combination with the clip provides a releasable attachment of the ultrasound probe to the base when the ultrasound probe is placed into the frame. In some cases, the hinge mechanism includes a first pin on one side of the frame and a second pin on an opposite side of the frame. The first and second pins are configured to engage corresponding slots defined in or on the ultrasound probe. In some cases, the hinge mechanism includes a first slot on one side of the frame and a second slot on an opposite side of the frame, wherein the first and second slots are configured to engage corresponding pins defined in the ultrasound probe.

An example of an ultrasound system includes an ultrasound probe and a probe-human interface. The ultrasound probe has a transducer configured to transmit and receive ultrasound signals. The probe-human interface includes a base having an attachment mechanism configured to secure the base to a body of a patient, and a center opening that is sized and arranged to receive the ultrasound probe. The center opening provides access to the patient for the transducer to transmit and receive ultrasound signals to and from the patient. The probe-human interface further includes a clip configured to receive a portion of the ultrasound probe when the ultrasound probe is placed into the probe-human interface. The clip is configured to secure the ultrasound probe to the probe-human interface.

In some cases, the clip is biased toward the center opening to bear against the ultrasound probe and provide a selective positional engagement of the ultrasound probe within the probe-human interface. In some cases, the ultrasound probe has a gear protrusion with a plurality of gear teeth that are arranged on the gear protrusion to engage with corresponding pawl teeth of the clip. The gear teeth and the pawl teeth may be arranged on the ultrasound probe and the probe-human interface, respectively, for selective engagement such that an orientation of the ultrasound probe relative to the patient is adjustable.

In some cases, the ultrasound system further includes a hinge mechanism providing a rotational engagement of the ultrasound probe with the probe-human interface for selective positioning of the ultrasound probe within the probe-human interface. The hinge mechanism may include one or more pins that engage one or more corresponding slots defined respectively in the ultrasound probe and the probe-human interface.

In some cases, the clip is a flexible ratchet clip that facilitates an engagement of the ultrasound probe with the probe-human interface. When the ultrasound probe is placed into the probe-human interface and a desired orientation of the ultrasound probe is achieved, a ratchet mechanism secures the ultrasound probe in place within the probe-human interface, thus positioning the ultrasound probe in a desired position with respect to the patient.

By way of example, also disclosed herein is a method for ultrasound examination. The method includes placing a probe-human interface on a body of a patient. The probe-human interface includes clip and a base. The base has a center opening that is sized and arranged to receive an ultrasound probe in a releasable engagement. The center opening provides access for a transducer of the ultrasound probe to transmit and receive ultrasound signals to and from the body of the patient. The method further includes affixing the probe-human interface to the body of the patient; placing an ultrasound probe into the probe-human interface such that the ultrasound probe is secured by the clip to the base of the probe-human interface; and removing the ultrasound probe from the probe-human interface while the probe-human interface remains affixed to the body of the patient. The probe-human interface is positioned on the body of the patient such that, each time the ultrasound probe is placed into the probe-human interface, the ultrasound probe is secured in a consistent position relative to the patient.

In some cases, the method includes affixing the probe-human interface to the body of the patient by exposing an adhesive on the base and adhering the base to the body of the patient. In some cases, placing the ultrasound probe into the probe-human interface includes hingedly coupling the ultrasound probe to the base and rotating the ultrasound probe into position relative to the clip to be secured by the clip to the base. In some cases, before affixing the probe-human interface to the body of the patient, the method includes placing the ultrasound probe into the probe-human interface and obtaining one or more ultrasound images of the patient while repositioning the probe-human interface on the patient until a desired ultrasound image is obtained, after which the probe-human interface is affixed to the body of the patient whereby the ultrasound probe is removable and reattachable to the probe-human interface and thus obtain consistent ultrasound images of the patient.

All illustrations shown in the drawings are provided for describing examples of the present disclosure and are not intended to limit the scope of the claimed invention. Moreover, aspects of the disclosure have been explained in relation to the illustrated embodiments, but it should be understood that modifications and variations can be made therein without departing from the spirit and scope of the disclosure.

1. A probe-human interface, comprising:
a base with an attachment mechanism configured to secure the base to a body of a patient;
a frame connected to the base, the frame having a center opening sized and arranged to receive an ultrasound probe and provide access to the patient for the ultrasound probe to send and receive ultrasound signals to and from the patient;
a clip configured to receive a portion of the ultrasound probe when the ultrasound probe is placed into the frame, wherein the clip is configured to secure the ultrasound probe to the frame.

2. The probe-human interface of claim 1, wherein the clip is a flexible ratchet clip that is coupled to the base at an angle relative to the base, and the angle of the clip facilitates an engagement between the clip and the ultrasound probe when the ultrasound probe is placed into the frame.

3. The probe-human interface of claim 2, wherein the clip is biased toward the center opening such that when the ultrasound probe is placed into the frame, the clip bears against a housing of the ultrasound probe.

4. The probe-human interface of claim 2, wherein the clip has one or more laterally-extending ratchet elements that are configured to engage with a protrusion on the ultrasound probe.

5. The probe-human interface of claim 3, wherein the one or more laterally-extending ratchet elements include pawl teeth that are configured to engage with corresponding gear teeth on the protrusion of the ultrasound probe.

6. The probe-human interface of claim 1, wherein the clip is configured to secure the ultrasound probe within the frame at a plurality of positions with respect to the base.

7. The probe-human interface of claim 1, further comprising a hinge mechanism coupled to the frame, wherein the hinge mechanism in combination with the clip provides a releasable attachment of the ultrasound probe to the base when the ultrasound probe is placed into the frame.

8. The probe-human interface of claim 7, wherein the hinge mechanism includes a first pin on one side of the frame and a second pin on an opposite side of the frame, wherein the first and second pins are configured to engage corresponding slots defined in the ultrasound probe.

9. The probe-human interface of claim 7, wherein the hinge mechanism includes a first slot on one side of the frame and a second slot on an opposite side of the frame, wherein the first and second slots are configured to engage corresponding pins defined in the ultrasound probe.

10. An ultrasound system, comprising:
an ultrasound probe with a transducer configured to transmit and receive ultrasound signals; and
a probe-human interface including a base having (1) an attachment mechanism configured to secure the base to a body of a patient, and (2) a center opening sized and arranged to receive the ultrasound probe, wherein the center opening provides access to the patient for the transducer to transmit and receive ultrasound signals to and from the patient;
the probe-human interface further including a clip configured to receive a portion of the ultrasound probe when the ultrasound probe is placed into the probe-human interface, the clip being configured to secure the ultrasound probe to the probe-human interface.

11. The ultrasound system of claim 10, wherein the clip is biased toward the center opening to bear against the ultrasound probe and provide a selective positional engagement of the ultrasound probe within the probe-human interface.

12. The ultrasound system of claim 10, wherein the ultrasound probe has a gear protrusion with a plurality of gear teeth that are arranged on the gear protrusion to engage with corresponding pawl teeth of the clip.

13. The ultrasound system of claim 12, wherein the gear teeth and the pawl teeth are arranged on the ultrasound probe and the probe-human interface, respectively, for selective engagement such that an orientation of the ultrasound probe relative to the patient is adjustable.

14. The ultrasound system of claim 10, further comprising a hinge mechanism providing a rotational engagement of the ultrasound probe with the probe-human interface for selective positioning of the ultrasound probe within the probe-human interface.

15. The ultrasound system of claim 14, wherein the hinge mechanism comprises one or more pins that engage one or more corresponding slots defined respectively in the ultrasound probe and the probe-human interface.

16. The ultrasound system of claim 10, wherein the clip is a flexible ratchet clip that facilitates an engagement of the ultrasound probe with the probe-human interface, wherein when the ultrasound probe is placed into the probe-human interface and an orientation of the ultrasound probe is achieved, a ratchet mechanism secures the ultrasound probe in place within the probe-human interface, thus positioning the ultrasound probe in a desired position with respect to the patient.

17. A method for ultrasound examination, comprising:
placing a probe-human interface on a body of a patient, wherein the probe-human interface includes clip and a base, the base having a center opening that is sized and arranged to receive an ultrasound probe in a releasable engagement, wherein the center opening provides access for a transducer of the ultrasound probe to transmit and receive ultrasound signals to and from the body of the patient;
affixing the probe-human interface to the body of the patient;
placing an ultrasound probe into the probe-human interface such that the ultrasound probe is secured by the clip to the base of the probe-human interface; and
removing the ultrasound probe from the probe-human interface while the probe-human interface remains affixed to the body of the patient,
wherein the probe-human interface is positioned on the body of the patient such that, each time the ultrasound probe is placed into the probe-human interface, the ultrasound probe is securable in a consistent position relative to the patient.

18. The method of claim 17, wherein affixing the probe-human interface to the body of the patient includes exposing an adhesive on the base and adhering the base to the body of the patient.

19. The method of claim 17, wherein placing the ultrasound probe into the probe-human interface includes hingedly coupling the ultrasound probe to the base and rotating the ultrasound probe into position relative to the clip to be secured by the clip to the base.

20. The method of claim 17, wherein before affixing the probe-human interface to the body of the patient, the method includes placing the ultrasound probe into the probe-human interface and obtaining one or more ultrasound images of the patient while repositioning the probe-human interface on the patient until a desired ultrasound image is obtained, after which the probe-human interface is affixed to the body of the patient whereby the ultrasound probe is removable and reattachable to the probe-human interface and able to obtain consistent ultrasound images of the patient.
